(19) United States
(12) Patent Application Publication
(10) Pub. No.: US 2022/0192281 A1
(43) Pub. Date: Jun. 23, 2022

(54) Medical assistive devise holder for life support and life sustaining lines and devices

(71) Applicant: Sylvia Marie Houston, Loganville, GA (US)

(72) Inventor: Sylvia Marie Houston, Loganville, GA (US)

(21) Appl. No.: 15/445,909

(22) Filed: Feb. 28, 2017

(51) Int. Cl. A41D 1/215

(52) U.S. Cl. CPC A41D 1/215; A41D 2300/33; A41D 2400/482; A41D 2600/00; A41D 2300/326

(57) ABSTRACT

This Medical Assistive Device Holder for Life Support and Life Sustaining Lines and Devices secures multiple tubes at the same time to prevent dislodging them from the patient making safe handling of an infant possible in Skin-to-Skin Contact while maintaining an intact delivery system whether, IV, ET, NG, PIC/Central lines, O₂ delivery and monitoring lines, etc. which makes it unique to preemies and NICU infants facilitates the additional benefits, hemodynamically and neurologically, for infant and mother in Skin-to-Skin Contact strengthening the immune system and neuro development often stunted in the premature or touch deprived infants not to mention reduction in stress levels (and hormones) of mother and infant. This Medical Assistive Device Holder for Life Support and Life Sustaining Lines and Devices is easily adapted for toddler and child through adult applications.

### RELATED APPLICATIONS

The present application is related to United States patent number US 20110087171 A1, published Apr. 14, 2011, for METHOD AND APPARATUS TO SECURE LIFE SUPPORT LINES TO AN INFANT DURING NEONATAL CARE, by Jennifer Bracci, included by reference herein.

The present application is related to United States patent number US 20060206978 A1, published Sep. 21, 2006, for MEDICAL GARMENTS FOR ASSISTING IN SKIN-TO-SKIN HOLDING OF INFANTS IN NEONATAL INTENSIVE CARE UNITS, by Shandin Hilton and Pamela Sprague, included by reference herein.

The present application is related to United States patent number US 2015/0119845 A1, publication date, Apr. 30, 2015, for SECURING DEVICE FOR MEDICAL LINES, by Steve A. Collins and Cynthia G. Collins, included by reference herein.

The present application is related to U.S. Pat. No. 5,755,225 A, publication date May 26, 1998, for MEDICAL TUBE-RETAINING DEVICE, by Teresa H. Hutson, included by reference herein.

The present application is related to United States patent number US 20110108594 A1, issued May 12, 2011, for MEDICAL GARMENT FOR SKIN-TO-SKIN CARE AND METHODS OF USE, by Amanda Marie Davis, included by reference herein.

The present application is related to U.S. Pat. No. 7,850,595 B2, published Dec. 14, 2010, for TRANSFERRING AND HOLDING DEVICE FOR HIGH-RISK NEONATAL INTENSIVE CARE UNIT (NICU) PATIENTS, by Robert D. White, included by reference herein.

The present application is related to United States patent number US 20150089710 A1, published Apr. 2, 2015, for SKIN-TO-SKIN CARE GARMENT, by Matthew C. Halverstadt, Katie A. Halverstadt and Hudlo, LLC, included by reference herein.

The present application is related to United States patent number US D752855 S1, published Apr. 5, 2016, for INFANT CARRIER GARMENT, by Matthew C. Halverstadt, Katie A. Halverstadt and Hudlo, LLC, included by reference herein.

The present application is related to U.S. Pat. No. 8,763,562 B2, published Jul. 1, 2014, for WEARABLE MEDICAL TUBING AND CABLING CONTAINMENT HARNESS, by Theresia Trevan Fladl, Thomas Lloyd Bellaire, Ernie Janzen and British Columbia Institute of Technology, included by reference herein.

The present application is related to United States patent number U.S. Pat. No. 4,168,544A, issued Sep. 25, 1979, for ARTICLE HOLDING SYSTEM, by Robert A. Kallman, included by reference herein.

The present application is related to United States patent number U.S. Pat. No. 4,688,270A, issued Aug. 25, 1987, for GARMENT FOR SHEILDING LINES CONNECTED TO A PATIENT DURING INVASIVE THERAPY, by Patricia J. Denicola, Patricia A. Gorgone and Children's Hospital Medical Center Cinncinnati Ohio 45229 A CORP OF OHIO, Cincinnati Children's Hospital Medical Center, included by reference herein.

The present application is related to United States patent number U.S. Pat. No. 5,897,519A, issued Apr. 27, 1999, for INTRAVENOUS SECURING DEVICE AND SECONDARY WOUND DRESSING, by Barry F. Sheshol, Marshall P. Reich, George. Glumac and Tapless. TECHNOLOGIES, Inc, Tapeless Tech Inc, included by reference herein.

The present application is related to U.S. Pat. No. 6,460,187 B1, published Oct. 8, 2002, for MEDICAL CLOTHING, by Marilyn R. Siegel, included by reference herein.

### FIELD OF THE INVENTION

This present invention relates to and, more particularly, to a medical assistive device holder to secure life support and life sustaining lines and devices. This present invention can be used with one or more Mom's Second Skin Swaddles, the Kango Wrap, the Mama Swaddle and the Cami Carrier, all designed to hold an infant in, or out of, Skin-to-Skin Contact. This present invention can be used for hands-free breast pumping, single or multiple births, premature, low birth weight or full term medically compromised infants and is not restricted for “infant only” applications. This present invention can also be used with the Comfort Band Tie or other securement garment/s to position and secure all life support lines. This present invention is freely adjustable regardless of body shape or size and has application with regard to large and small animal use. However, modifications and variations to those skilled in the art allow for alterations and variations to occur without departing from the original scope and use.

Other embodiments of this invention will be apparent to those skilled in the art from the consideration of the specification and practice of the invention disclosed herein. It is intended that the specification and examples be considered as exemplary only and that modifications may be made to the described embodiments without departing from the spirit and scope of the invention as defined in the Claims.

### BACKGROUND OF THE INVENTION

Research has shown multiple benefits of holding an infant, especially in Skin-to-Skin Contact, for both the newborn and mother. However, accomplishing this can be complicated in infants with special needs requiring life support and life sustaining measures. Medical staffs have reported two important concerns with infants being held, especially in Skin-to-Skin Contact, 1) there was no garment or assistive medical apparatus that could securely hold and position life support or life-sustaining lines and devices in place without threat of displacing them or extubation and, 2) pathogen cross-contamination of medical lines in Skin-to-Skin Contact. Since there exists a strong potential for an unexpected crisis to develop in the hospitalized medically compromised infant, the need for a securement garment/apparatus for medical lines, tubing and devices that can be rapidly released from the wearer (adult holding the infant in Skin to Skin Contact) to transfer with the infant for emergency intervention without the threat of dislodging life support/life-sustaining lines and medical devices was urgent.

This present invention serves to secure and stabilize life support lines, in the medically fragile/compromised infant with extended uses applicable to medical assistive devices employed to monitor body function/s, administer medications, IV fluids, O₂ and/or provide diagnostic evaluation of a patient when assistive medical devices are necessary to establish and maintain homeostasis. Specifically, this invention accommodates medical devices commonly used in the critical or compromised infant to include but not limited to various central lines, gastronomy tubes, Ostomy bags, Endotracheal tubes, a multiplicity of medical monitors and other supportive or life-support devices.

An increasing number of patients are born with critical medical issues requiring the continuous use of assistive medical devices that perform and support vital bodily and life-sustaining functions beyond the hospital setting. The number of medical devices needed to sustain life and assist in proper body function is determined by the diagnosed medical condition; further the ability to secure the necessary medical device/s and various life-support lines in place becomes critical to effective and successful outcomes whether the device/s will be necessary on a temporary or permanent basis.

Unfortunately, the use of, and need for, such assistive medical devices and life-support lines are required most in the premature underdeveloped infant or an infant born with improperly formed or malfunctioning organs/systems. In these infants, normal routes for hydration, nutrition, elimination and/or adequate lung, heart, immune and gastrointestinal system function is compromised requiring medical assistive devices to be employed to sustain life. The need for such devices can be temporary or permanent depending on the body's ability to restore function as the infant matures or successful surgical intervention corrects the problem or permanent if medical/surgical intervention is unsuccessful or a progressive degenerative condition is present.

Common life-sustaining lines such as Gastronomy tubes, central venous catheter, PICC lines (used for long-term intravenous antibiotics, nutrition or medications, and for blood draws), Jackson Pratt lines (a closed-suction medical device commonly used as a post-operative drain collecting bodily fluids from surgical sites), Ostomy (colostomy/ileostomy), various catheters; assorted monitoring devices, such as cardiac, pacemakers (internal or external) and O₂ sensors (internal and external) can present increased infection potential if the site/s are not kept clean and secure. Invasive lines can become easily dislodged if they cannot be held securely in place, which can cause additional medical and surgical procedures to correct placement increasing infection potential exponentially. This potential makes the need for a garment/line holder invention necessary to effectively and securely hold all lines and medical devices in place protecting sites into and exiting from an infant's body. This present invention ensures secure hold and also creates an additional safety barrier inhibiting cross contamination from mother's skin to the infants life support/life-sustaining lines, tubing or medical devices significantly decreasing pathogens from coming into contact with entry sites of catheters/medical devices that penetrate the skin of the body. This present invention is easily adaptable to a growing infant accommodating all stages of growth from infancy to adult. This present invention can also be altered adding various pockets to hold monitoring devices securely in place as the infant grows from infancy to adulthood including but not limited to insulin pumps, O₂ delivery system and various monitoring or medication administration devices.

The unique design of this present invention is easily transferred with an infant to and from an incubator or bassinet without disturbing life support, life sustaining lines or medical devices in a hospital or home setting. As the infant grows the line holder can be secured in various ways with a multiplicity of attachments to accommodate a changing body shape from infant to toddler, child, pre-teen, teen and adult, providing secure hold and protection while preserving the privacy and dignity of the wearer. The breathable, flexible and water friendly fabric of this present invention allows for adaptability to all stages of growth, body shape, activity levels and sweating without losing its shape or hold and cleans easily drying quickly.

This present invention also has application for acquired disease processes, including traumatic, in the adult population and can be easily altered to accommodate individualistic needs to include pocket additions, attachments and varied body positioning on the wearer to accommodate multiple medical devices, lines, tubing, catheters and monitoring devices with secure hold and privacy.

Thus, the Medical Assistive Device Holder for Life Support and Life Sustaining Lines and Devices was designed and created. The Medical Assistive Device Holder for Life Support and Life Sustaining Lines and Devices has met the challenge providing an opportunity for mothers to safely hold their special needs or medically compromised infant(s) in or out of Skin-to-Skin Contact. For Skin-to-Skin Contact, when used in conjunction with one or more Mom's Second Skin Swaddles, the Kango Wrap, the Mama Swaddle, the Cami Carrier, the Comfort Band Tie or other securement garments, the infant(s) and all life support and life sustaining lines and medical devices are safely secured with correct placement maintained. The Medical Assistive Device Holder for Life Support and Life-Sustaining Lines and Devices easily adapts for use in the NICU, a hospital or birthing center setting throughout all phases of life addressing a multitude of medical needs whether in the hospital, home, school, workplace, rehabilitation or geriatric setting. In some applications, when used in childhood to adulthood, the Medical Assistive Device Holder for Life Support and Life Sustaining Lines and Devices can be secured in place with a multiplicity of closures to include but not limited to anchor, D-ring, slide buckles, slide release buckles, as well as varying closures from the group consisting of hook and loop closures, hook and eye closures, snaps, zippers, magnets, and the like.

This present invention design secures lines tubing and life support life sustaining devices enabling an infant(s) to be held Skin-to-Skin in the neonatal intensive care unit and beyond preventing extubation, the number one problem with pre-term infants being held Skin-to-Skin. The use of this Line Holder greatly reduces that possibility while simultaneously organizing all lines and tubing entering and exiting the medically compromised infant(s) body, which facilitates frequency and duration of safe infant hold by a parent or hospital staff. This present invention works independently and easily adjusts to fit all body shapes, sizes or garment(s) worn allowing a custom fit with secure hold and comfort for extended lengths of time without pathogen contamination of lines or devices by touching the wearer's skin.

With the use of the Medical Assistive Device Holder for Life Support and Life Sustaining Lines and Devices the benefits of Skin-to-Skin Contact, as established by over 4,000 research studies and articles, can be realized in the most medically compromised of infants.
*Ludington-Hoe SM, OHIO WAR ON INFANT MORTALITY: The Newborn Kangaroo Care Project.

It was presented at the KMC Acceleration Meeting of Maternal, Newborn and Child Health, the Bill and Melinda Gates Foundation, Save the Children Saving Newborn Lives and others on Oct. 21-22, 2013 in Istanbul, Turkey:
“Kangaroo Mother Care (KMC) was initially developed in Bogota 35 years ago when there was a shortage of incubators for preterm babies. When practiced in hospitals, it consists of continuous skin-to-skin contact, establishing breast feeding, early discharge, and close-follow-up. KMC is widely recognized to promote physiological stability, facilitate breastfeeding, keep a baby warm, reduce the risk of serious infections and reduce the mortality of hospitalized, stable premature infants by about 50 percent. This practice is also a wonderful way to promote bonding between infants and their parents, and may have lasting neuro-developmental benefits.
In spite of these benefits, however, some key challenges have prevented KMC from being adopted widely across the globe. First, even where mothers receive training on KMC, socio-cultural, resourcing, and experiential barriers—such as a lack of support for the mother from the family and community—make practicing KMC difficult. Second, guidelines for KMC recommend initiation of the practice in the health facility, which means that the large portion of infants born outside of these health facilities may not have access to this practice. Even for mothers who deliver their babies in facilities, we have a long way to go to ensure that most premature babies have access to quality KMC services and support.
If universal KMC coverage was achieved, it is estimated that it could save the lives of more than 450,000 preterm newborns each year. However, despite global recognition of its inherent benefits and potential to improve newborn health, KMC is still struggling to catch on—with less than one percent coverage globally and no universally agreed upon indicator to measure uptake or impact.”
*Claeson, M., Darmstadt, G., Engmann, C., and Wall, S., (2013) Accelerating Global Practice of Kangaroo Mother Care
   Method and Apparatus to Secure Life Support Lines to an Infant During Neonatal Care—Patent No.: US 20110087171 A1
   Medical Garments for Assisting in Skin-to-Skin Holding of Infants in Neonatal Intensive Care Units—Patent No.: US 20060206978 A1
   Securing Device for Medical Lines—Patent No.: US 2015/0119845 A1
   Medical Tube-Retaining Device—Patent No.: U.S. Pat. No. 5,755,225 A
   Medical Garment for Skin-to-Skin Care and Methods of Use—Patent No.: US 20110108594 A1
   Transferring and Holding Device for High-Risk Neonatal Intensive Care Unit (NICU) Patients—Patent No.: U.S. Pat. No. 7,850,595 B2
   Skin-to-Skin Care Garment—Patent No.: US 20150089710 A1
   Infant Carrier Garment—Patent No.: U.S. D752855 S1
   Wearable Tubing and Cabling Containment Harness—Patent No.: U.S. Pat. No. 8,763,562 B2
   Article Holding System—Patent No.: U.S. Pat. No. 4,168,544A
   Garment for Shielding Lines Connected to a Patient During Invasive Therapy—Patent No.: U.S. Pat. No. 4,688,270A
   Intravenous Securing Device and Secondary Wound Dressing—Patent No.: U.S. Pat. No. 5,897,519A
   Medical Clothing—Patent No.: U.S. Pat. No. 6,460,187 B1

### Related Applications as Applied to Medical Assistive Device Holder for Life Support and Life Sustaining Lines and Devices:

Method and Apparatus to Secure Life Support Lines to an Infant during Neonatal Care—Patent No.: US 20110087171 A1
This application does not provide for or provide secure support of an infant in Skin-to-Skin Contact
Infant hold in this application is dependent upon wearer
Fastener of this application in a one site location unable to provide flexibility in multi-life support/sustaining line placement—e.g.
Endotracheal tube is not securable from inferior placement—most commonly used due to condensation collection in tubing
This application is limited in the number of lines that be accommodated
This application clips to clothing in a manner that can slip risking dislodging of life support/sustaining lines
This application has a potential for kinking of life support/sustaining lines

### Medical Garments for Assisting in Skin-to-Skin Holding of Infants in Neonatal Intensive Care Units—Patent No.: 20060206978 A1

Side openings can cause contamination of support lines increasing infection potential through contact with the wearer's skin
Side string ties connecting the front and back of the garment which can become untied, does not conform to the body of the wearer or infant held Skin-to-Skin and cannot provide secure support allowing slippage which can dislodge life support lines and compromise airway patency (improper head alignment, extubation or airway blockage from the dislodged endotracheal tube, etc.)
With a collar at the neck, does not leave the chest area where the head of the infant would be visible to observe infant positioning and life support lines without opening the garment
Accessory “hook-and-loop fastener, a button, a snap, a clip, a buckle, a strap, a tie string, or combinations thereof” do not provide secure support of the infant and can be cumbersome
Front opening with attachment closing fastener(s) can contact infant's skin causing irritation to fragile tissue; can apply excessive pressure resulting in related injury or respiratory/airway compromise
Correct Skin-to-Skin Contact utilizes the chest area only to gain greatest benefit without airway compromise
Garment does not provide secure hold of the infant nor maintains correct positioning
Correct Skin-to-Skin Contact is dependent upon the wearer holding the infant in the correct position with attention to life support lines. If the wearer falls asleep, expected in Skin-to-Skin Contact, the infant is no longer secure and can slip dislodging life support lines and airway patency

### Securing Device for Medical Lines—Patent No.: US 2015/0119845 A1

This application requires a line coupling device, a multiport, extension tubing and an adhesive backed base secured to the patient's skin
This application does not secure Endotracheal tubing
This application does not secure life-monitoring leads (i.e., EKG, thermomonitor, pulse oximeter, etc.)
   Medical tube-retaining device—Patent No.: U.S. Pat. No. 5,755,225 A
This application is a device for holding a medical tube, such as a nasal Endotracheal tube or IV catheter, in fixed position on the body near the tube insertion site and includes a clamp restricting sliding movement of the tube within the clamp which can cause displacement of life support/life sustaining lines with spontaneous reflex movement of the infant
This application comprises a flexible patch with an adhesive coating on its back surface for adhering the tube-retaining device directly to the skin of the patient which can be repositioned however, with each placement the hold is significantly weakened
This application attaches to the body of the patient by an adhesive at the entry point into the body which can facilitate allergic reactions and tears compromising the integrity of fragile skin in medically compromised infant/s

### Medical Garment for Skin-to-Skin Care and Methods of Use—Patent No.: US 20110108594 A1

This application is for single birth use and utilizes ties as its primary line and tubing securement. It is therefore limited in its application when a multiplicity of lines of varying sizes is in use; the greater the number of lines the less secure the hold
Line securing ties of this invention does not allow for correct positioning of the Endotracheal tube for proper condensation collection thereby compromising the airway, further there is no provision for securing umbilical, central or distally located life support/life sustaining line/s
   Transferring and Holding Device for High-Risk Neonatal Intensive Care Unit (NICU) Patients—Patent No.: U.S. Pat. No. 7,850,595 B2
This application is a transferring and holding device for holding infants having lifelines, including IV tubes, monitor wire leads, and ventilator tubes, connected thereto, the transferring and holding device includes a base and spaced-apart pegs into which the lines fit between the pegs and others are wrapped around the pegs for securing the lifeline to the securing—mechanism may include a cap Infant cannot be transferred independent of the device
This application is cumbersome—bulky, weighted and awkward in attempting Skin-to-Skin Contact due to the size and mechanisms in place to secure lines
This application does not allow for complete Skin-to-Skin Contact due to attachment fasteners
This application does not allow for contact with the infant's back with device in place
This application maintains supine infant positioning—prone is needed in Skin-to-Skin Contact

### Skin-to-Skin Care Garment—Patent No.: US 20150089710 A1

This application does not secure life support/sustaining lines Direct contact of infant's support lines with wearer of this application can cause contamination of support lines increasing infection potential through contact with the wearer's skin

### Infant Carrier Garment—Patent No.: US D752855 S1

This application does not secure life support/sustaining lines Direct contact of infant's support lines with wearer of this application can cause contamination of support lines increasing infection potential through contact with the wearer's skin
   Wearable Medical Tubing and Cabling Containment Harness—Patent No.: U.S. Pat. No. 8,763,562 B2
This application has to be worn by the patient suggested age range 3-36 months—not applicable in the preemie, premature or infants less than 3 months or those with body malformations/missing/compromised limbs since the device is supported by the shoulder, torso and thigh
As this application is worn by the patient—not possible in the NICU preventing medical access and visible assessment, especially due to the small size of the medically compromised infant and the multiple lines attached to the infant's chest, umbilicus and abdominal torso (i.e., ECG leads, central lines, etc.)
Multiplicity of fastener(s) styles in use in this application increase device failure
Hooks and eyes of this application, if metal can interfere with cardiopulmonary electro resuscitation
   Article Holding System—Patent No.: U.S. Pat. No. 4,168,544A
This application is an outside garment with an attachment for carrying items through the use of a shoulder epaulette style fastener
This application is not applicable for in hospital use
This application does not secure life support/sustaining lines
This application is not applicable for infant or NICU use
   Garment for Shielding Lines Connected to a Patient During Invasive Therapy—Patent No.: U.S. Pat. No. 4,688,270A
This application must be worn by the infant
This application is not applicable for the preemie, low birth weight or medically fragile infant
This application is impractical in the NICU
This application does not provide safe Skin-to-Skin Contact in the medically compromised infant
Correct infant hold in this application is dependent upon wearer
This application applies pressure on a fragile body, prevents maximum patient surface area visualization and delays emergency medical intervention access
   Intravenous Securing Device and Secondary Wound Dressing—Patent No.: U.S. Pat. No. 5,897,519A
This application is an intravenous securing device and secondary wound dressing for holding intravenous tubing in place
This application is not applicable for infants, especially preemie or NICU infants since multiple life support/sustaining lines are in use
This application is used 1:1 thereby requiring multiple devices to support multiple life support/sustaining lines in use with a medically compromised infant
   Medical Clothing—Patent No.: U.S. Pat. No. 6,460,187 B1
This application is a medical gown, Multipurpose medical clothing accommodates medical appliances attached to a patient, affords privacy, and thus encourages the patient to be up and ambulatory
This application is a garment for an adult
Not for infant application
This application is not applicable in a NICU setting

### SUMMARY OF THE INVENTION

In accordance with the present invention, there is provided a device that allows for safe and correct positioning of an infant held Skin to Skin providing secure hold of all life support and life sustaining lines and devices traditionally utilized in the medically compromised infant to include but not limited to Central Lines, PICC Lines, Umbilical Catheter/s, Intravenous Lines, Blood Pressure and Cardiopulmonary Monitoring Leads, Pulse Oximeter, Thermomonitoring, Suction Device, C-Pap, Endotracheal Tube, Mechanical Ventilator and other Oxygen Delivery or Invasive Therapy Lines.

The Medical Assistive Device Holder for Life Support and Life Sustaining Lines and Devices is easily adapted to all body shapes and sizes without restriction, is not limited to a fixed location on the body of a wearer and is applicable in multiple births with the addition of a second Medical Assistive Device Line Holder to the wearer on the opposite side of the body from the sibling.

The Medical Assistive Device Holder for Life Support and Life Sustaining Lines and Devices can be transferred with an infant should emergency medical intervention be needed without compromising, disturbing or dislodging life support or life sustaining line/s, device/s or catheter/s placement and/or delivery capabilities.

The Medical Assistive Device Holder for Life Support and Life Sustaining Lines and Devices is held in place by a securement garment where both ends of the Medical Assistive Device Line Holder are tenable. Its secure placement is best achieved with one or more Mom's Second Skin Swaddles, the Kango Wrap, the Mama Swaddle, Cami Carrier, or the Comfort Band. Tie, especially in multiple birth applications, but is not restricted to these securement garments alone. This present invention is applicable for infants classified as premature through large birth weight infants, multiple birth medically compromised infants including conjoined twins.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a front view of Medical Assistive Device Holder for Life Support and Life Sustaining Lines and Devices on the shoulder with hook and loop closures 3, 4, 5, 6, 7, 8 and 9 with the Mom's Second Skin Swaddle 10 as a securement garment for the present invention.

FIG. 2 is a back view of the Medical Assistive Device Holder for Life Support and Life Sustaining Lines and Devices seam on the underside of the invention secured in place by Mom's Second Skin Swaddle from the back.

FIG. 3 is a back view of the Medical Assistive Device Holder for Life Support and Life Sustaining Lines and Devices secured in place by the Mom's Second Skin Swaddle.

FIG. 4 is a front view of the Medical Assistive Device Holder for Life Support and Life Sustaining Lines and Devices showing correct infant placement with hook and loop closures securing posterior ascending endotracheal tube and life support life and life sustaining lines with secure hold by Mom's Second Skin Swaddle.

FIG. 5 is a front view of the Medical Assistive Device Holder for Life Support and Life Sustaining Lines and Devices showing correct placement of infant and over the shoulder endotracheal tube and medical lines and tubing secured in place with hook and loop closures above and below.

FIG. 6 is a front view of step one in securing an over the shoulder endotracheal tube with hook and loop closure 4 and 6 of the Medical Assistive Device Holder for Life Support and Life Sustaining Lines and Devices

FIG. 7 is a front view of step 2 in securing an over the shoulder endotracheal tube with hook and loop closure 4 and 7 of the Medical Assistive Device Holder for Life Support and Life Sustaining Lines and Devices.

FIG. 8 is a front view of correct infant positioning, endotracheal tube and life line placement secured with hook and loop closures of the Medical Assistive Device Holder for Life Support and Life Sustaining Lines and Devices with Mom's Second Skin Swaddle providing safe hold in Skin to Skin Contact.

FIG. 9 is a front view illustrating versatility of the Medical Assistive Device Holder for Life Support and Life Sustaining Lines and Devices as used with the Kangaroo securement garment and waist sash.

FIG. 10 is a front view of the Medical Assistive Device Holder for Life Support and Life Sustaining Lines and Devices with horizontal body placement for securing medical devices, tubing and lines for child through adult applications adaptable for animal use as well.

FIG. 11 is a front view of the Medical Assistive Device Holder for Life Support and Life Sustaining Lines and with diagonal body placement.

### DESCRIPTION OF THE PREFERRED EMBODIMENT

The following detailed description represents the best currently contemplated modes for carrying out the invention. The description is not to be taken in a limiting sense, but is made merely for the purpose of illustrating the general principles of the invention.

This Medical Assistive Device Holder for Life Support and Life Sustaining Lines and Devices is designed to secure lines and tubing on a person when holding an infant in a special care unit with the need for lines, tubing and medical assistive devices and is secured in place with our patent pending Second Skin Swaddle/Mom's Second Skin, the Kango Wrap, Mama Swaddle, Cami Carrier, Comfort Band Tie or other securement garment/s. Multiple Medical Assistive Device Holders for Life Support and Life Sustaining Lines and Devices may be used to secure lines, tubing and medical assistive devices for one or more infants. This present invention is easily adapted for toddler and child through adult applications.

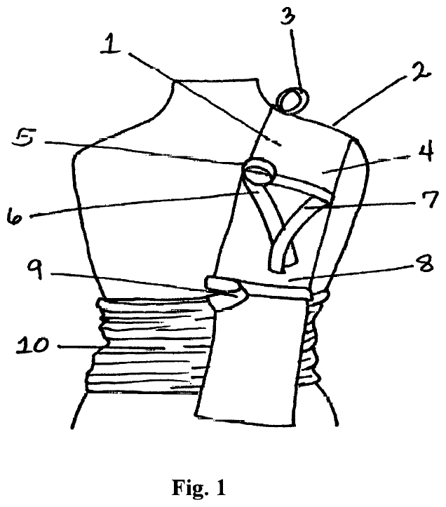

FIG. 1 shows the Medical Assistive Device Holder for Life Support and Life Sustaining Lines and Devices mounted on the sash body 1. The sash body 1 is cut 6″-10″ wide and 30″-40″ long depending on size of the wearer. The sash 1 is sewn with seam 2 forming two layers when flattened with seam 2 centered on the backside as shown in FIG. 1.

The Medical Assistive Device Holder for Life Support and Life Sustaining Lines and Devices of FIG. 1 consists of 3 tabs of hook and loop closures mounted on the sash body 1 with the first hook and loop closure positioned in the center of the length of the sash body 1 approximately 15-20″ from each end of sash and designed to be placed on the shoulder of wearer with shoulder hook and loop closure 3 sewn the width of sash body 1 measuring approximately 4-5″ in width for adult and modified width of approximately 3″ if worn by a child. Reinforced loop and hook closure 3 is sewn at one end and spans the width of the 3 plus 1-2″ with finished end tab.

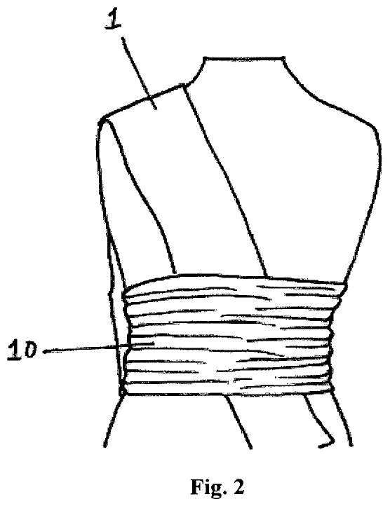

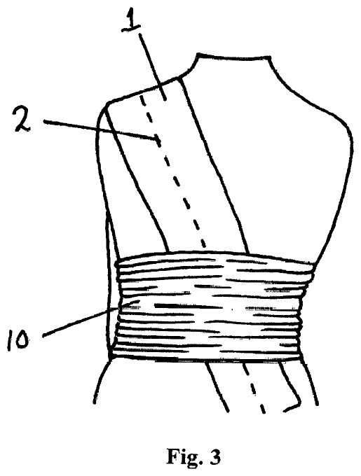

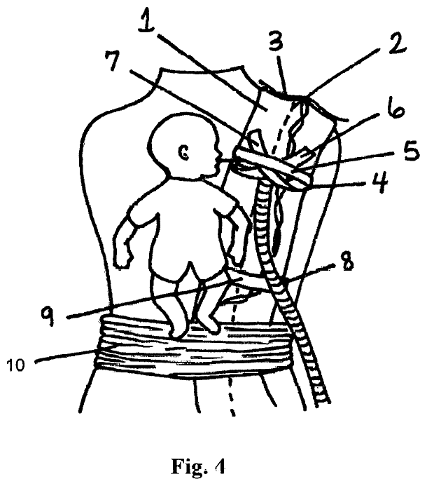

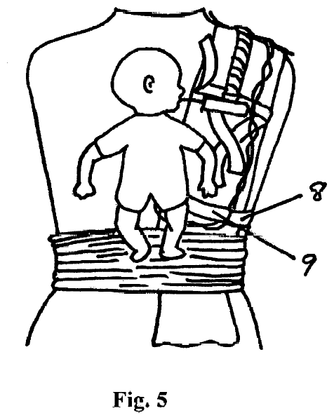

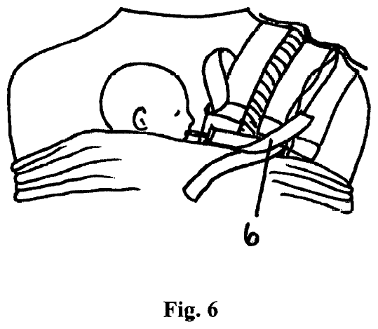

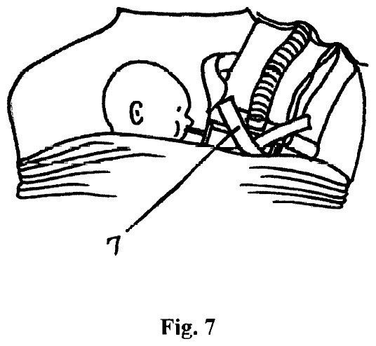

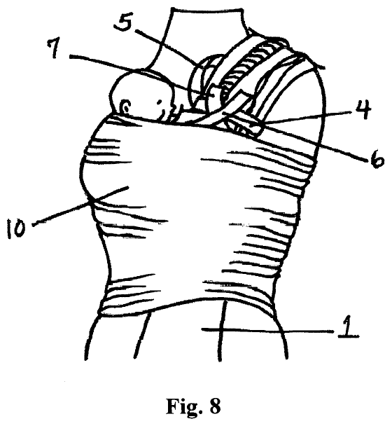

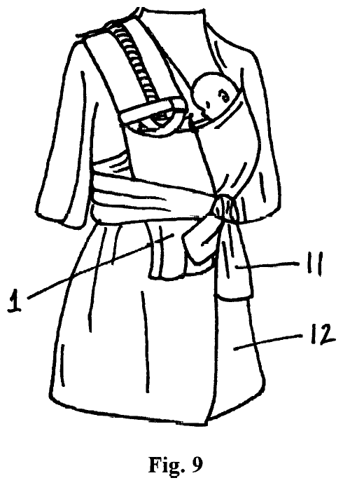

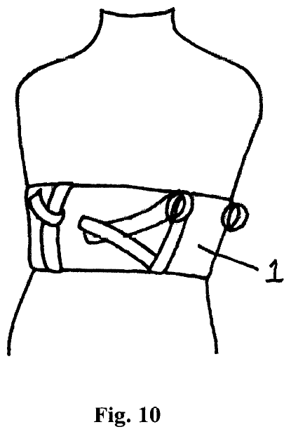

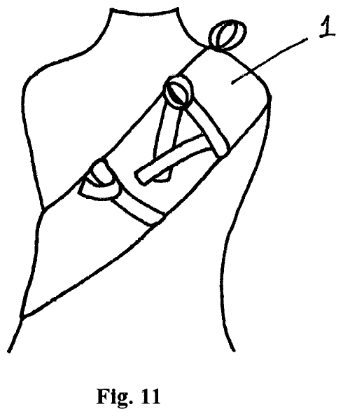

The second hook and loop closure is positioned approximately 3-5″ from the shoulder hook and loop closure depending on size of wearer. Hook closure 4 is positioned to be sewn the width of the line holder sash 1. Reinforced loop closure 6 and 7 are sewn in at an angle under projected ends of the hook closure 4 to form an X when spread. Hook closure 4 is sewn in place over angled strips 6 and 7 which measure approximately 8″ in length. Reinforced loop closure 5 is sewn to the end of hook closure 4 to span width of sash 1 plus 1-2″ with finished end tab. The lower hook and loop closure 8 and 9 is positioned approximately 4-6″ below the second hook and loop closure 4, 5, 6 and 7 with hook closure 8 sewn the width of sash body 1. The reinforced loop closure 9 is sewn to the end of hook closure 8 and spans the width of 8 plus 1-2″ with finished end tab. The Mom's Second Skin 10 is positioned at waist to secure line holder when in use.
FIG. 2 shows line holder sash body 1 in place over the shoulder with seam 2 positioned toward the underside joining the two layers of fabric and secured with the Mom's Second Skin Swaddle 10 in back.
FIG. 3 shows line holder sash 1 back secured with the Mom's Second Skin 10 in back.
FIG. 4 shows line holder supporting a respirator between hook and loop closure 4, 5, 6 and 7 with life support life sustaining lines secured by the lower hook and loop closure 8/9, second closure 4/5 and shoulder closure 3. The Mom's Second Skin 10 is positioned at waist to secure the line holder.
FIG. 5 shows loop closure 9 pulled up over life support lines and tubing secured on hook closure 8.
FIG. 6 shows loop closure 6 pulled up crossing over respirator tubing attached and secured on the opposite side to hook 4.
FIG. 7 shows loop closure 7 pulled up crossing over respirator tubing attached and secured on the opposite side to hook 4.
FIG. 8 shows an upward line respirator secured by the line holder. A securement garment, the Mom's Second Skin is securing the lower front of the line holder sash body 1. The lower lines are secured by hook and loop closure 8/9 under the securement garment, Mom's Second Skin. The tubing and lines are secured by hook 4 and loop closure 5 securing loop closures 6/7 on hook closure 4 and securing life support lines and tubing on hook closure 4 and shoulder hook and loop closure 3.
FIG. 9 shows line holder sash with mounted line holder secured with comfort band tie of the Kango Wrap garment.
FIG. 10 shows line holder sash 1 worn around the body in a horizontal fashion demonstrating versatility in holding medical devices, lines and tubing secured in place with a variety of closures accommodating individual needs from infancy through childhood to adulthood.
FIG. 11 shows line holder sash 1 worn diagonally across the body secured in place by a multiplicity of closures to accommodate individual needs and preferences to secure a variety of medical devices, lines or tubing.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters of Patent is presented in the subsequently appended claims.

What is claimed is:

1. A device, comprising:
Designed to secure multiple life support and life sustaining lines and devices in the medically comprised infant to include the premature, low birth weight and full term infant(s) in need of medical intervention to include the NICU when an adult is holding a medically comprised infant(s) in Skin to Skin Contact. The present invention is to be draped over the shoulder of an adult wearer and secured in place with our patent pending Second Skin Swaddle, or other securement garment(s). A device comprising:
a predetermined length and width of a strip of flexible woven or jersey-type fabric with slight stretch which adjusts and moves independently with the patient,
wherein said fabric is doubled along the length and seamed with a flat seam to form a two-ply sash,
on which is affixed through stitching or other methods of securement a plurality of adjustable, self-gripping, and re-sealable closures of a length and width to accommodate multiple sizes of life support and life sustaining lines and medical devices to said two-ply sash;
on which pockets may be formed through stitching the two tube layers together cutting appropriate opening to slide medical device to be supported into
said two-ply sash is designed to be draped over the shoulder of the wearer and secured in place with the patent pending Second Skin Swaddle or other securement garment(s)
ends of said two-ply sash may be joined to adapt for wear by toddler and child through adult applications with closures from the group consisting of anchor buckles, D-ring buckles, slide buckles, slide release buckles, and the like, or may be secured in place with varying closures from the group consisting of hook and loop closures, hook and eye closures, snaps, zippers, magnets, and the like and/or secured with the patent pending Second Skin Swaddle or other securement garment(s) by tucking the anterior and posterior sash ends between the wearer and the securement garment body.

2. The device of claim 1 may be knitted as single, tube-shaped piece and flattened to form a two-ply sash of a predetermined length and width.

3. The device of claims 1 and 2 consists of:
three or more separate adjustable, self-gripping, and re-sealable closures located from the center of the sash forward on sash front with the first adjustable, self-gripping, and re-sealable closure located in the proximity of the wearer's or otherwise as need for tubes, lines and devices running horizontally extending a predetermined length and width.
A plurality of adjustable, self-gripping, and re-sealable closures is located on the sash front in a predetermined attachment zone below the first closure extending a predetermined length and width with two attached loop tabs, or similar tabs, angularly secured in place under a horizontal hook base, or similar closures, to form an “X” thereby supporting and securing a tube or device when closed.
Additional adjustable, self-gripping, and re-sealable closure(s) as needed are located at predetermined lengths depending on wearer and lines, tubing and devices needing securement to meet diverse medical needs. All closures can be used to secure a multiplicity of life support and life sustaining line(s), lead(s), tubing(s) and device(s).
